(19) United States
(12) Patent Application Publication
(10) Pub. No.: US 2001/0051333 A1
(43) Pub. Date: Dec. 13, 2001

(54) Nuclear magnetic resonance methods for identifying sites in papillomavirus E2 protein

(75) Inventor: Brian J. Stockman, Kalamazoo, MI (US)

Correspondence Address: MUETING, RAASCH & GEBHARDT, P.A., P.O. BOX 581415, MINNEAPOLIS, MN, 55401, US

(73) Assignee: PHARMACIA & UPJOHN COMPANY

(21) Appl. No.: 09/829,872

(22) Filed: Apr. 10, 2001

Related U.S. Application Data
(60) Provisional application No. 60/197,459, filed on Apr. 17, 2000.
(60) Provisional application No. 60/211,055, filed on Jun. 13, 2000.
(60) Provisional application No. 60/268,444, filed on Feb. 13, 2001.

(51) Int. Cl. C12Q 1/70; G01N 33/53; C07K 14/00

(52) U.S. Cl. 435/5; 435/7.1; 530/350; 436/173

(57) ABSTRACT

Nuclear magnetic resonance methods for identifying sites in a DNA-binding and dimerization domain of a papillomavirus E2 protein are disclosed. Preferably the sites are ligand binding sites.

This application claims the benefit of U.S. Provisional Application Ser. No. 60/197,459, filed Apr. 17, 2000, No. 60/211,055, filed Jun. 13, 2000, and No. 60/268,444 filed Feb. 13, 2001, which are incorporated herein by reference in their entireties.

### BACKGROUND OF THE INVENTION

An important aspect in understanding the function of biochemical processes is the elucidation of the nature of the associations between various species including, for example, the associations between ligands and proteins. Such associations may be non-covalent, wherein juxtapositions are energetically favored by hydrogen bonding, van der Waals forces, or electrostatic interactions, or they may be covalent. When physical binding is being studied, a target molecule is typically exposed to one or more compounds suspected of being ligands, and assays are then performed to determine if complexes between the target molecule and one or more of those compounds are formed. Such assays, as are well known in the art, test for gross changes (e.g., size, charge, and mobility) in the target molecule that indicate complex formation.

Where functional changes are measured, assay conditions are established that allow for measurement of biological or chemical events related to the target molecule (e.g., enzyme catalyzed reaction and receptor-mediated enzyme activation). To identify an alteration, the function of the target molecule is determined before and after exposure to the test compounds.

Assays involving the use of nuclear magnetic resonance (NMR) techniques are also known. NMR techniques may be used, for example, in conjunction with other assay methods to assess hits identified from physical binding screens or functional assay screens. If ¹H, ¹³C, and/or ¹⁵N resonance assignments are known for the target as well as either a solution or X-ray crystallographic structure, then the binding site location of identified ligands can be determined using NMR techniques. As such, definitive resonance assignments of the target are required as a first step. A DNA-binding protein, E2, which is encoded by the papillomavirus and is involved in transcriptional regulation and viral replication, is one such target.

### SUMMARY OF THE INVENTION

In one aspect, the present invention provides a nuclear magnetic resonance method for identifying a site in a DNA-binding and dimerization domain of a papillomavirus E2 protein. In one embodiment, the method includes providing a first set of chemical shifts for atoms of a mixture including a ligand and the papillomavirus E2 protein, comparing the first set of chemical shifts to a second set of chemical shifts as listed in Table 1, and identifying at least a portion of the atoms that exhibit changes in chemical shifts, wherein the site includes the identified atoms. Preferably providing the first set of chemical shifts includes providing a mixture of the ligand and the papillomavirus E2 protein, allowing the ligand to interact with the papillomavirus E2 protein, obtaining a nuclear magnetic resonance spectrum of the mixture, and measuring chemical shifts of atoms from the spectrum. Preferably allowing the ligand to interact includes allowing the ligand and the protein to reach a binding equilibrium. Preferably the site is a ligand binding site. Preferably the papillomavirus E2 protein is encoded by the HPV-18 strain.

In another embodiment, the method includes providing a first ¹H-¹⁵N heteronuclear single quantum correlation spectrum of a mixture including a ligand and the papillomavirus E2 protein, comparing the first ¹H-¹⁵N heteronuclear single quantum correlation spectrum to a second ¹H-¹⁵N heteronuclear single quantum correlation spectrum as illustrated in FIG. 2, and identifying at least a portion of the amino acids having atoms that exhibit changes in chemical shifts, wherein the site includes the identified amino acids. Preferably providing the first spectrum includes providing a mixture of the ligand and the papillomavirus E2 protein, allowing the ligand to interact with the papillomavirus E2 protein, and obtaining a ¹H-¹⁵N heteronuclear single quantum correlation spectrum of the mixture. Preferably allowing the ligand to interact includes allowing the ligand and the protein to reach a binding equilibrium. Preferably the site is a ligand binding site. Preferably the papillomavirus E2 protein is encoded by the HPV-18 strain.

In another aspect, the present invention provides a machine-readable data storage medium including a data storage material encoded with nuclear magnetic resonance chemical shifts as listed in Table 1, wherein when a first set of chemical shifts is provided, the chemical shifts encoded on the data storage material are capable of being read by the machine to create a second set of chemical shifts, and the machine having programmed instructions that are capable of causing the machine to compare the first and second sets of chemical shifts to arrive at structural information.

In another aspect, the present invention provides a computer-assisted method for identifying a ligand binding site in a DNA-binding and dimerization domain of a papillomavirus E2 protein. The method includes providing a first set of nuclear magnetic resonance chemical shifts for atoms of a mixture including the ligand and the papillomavirus E2 protein, causing the first set of chemical shifts to be entered into memory of a computer, causing the computer to read a second set of chemical shifts as listed in Table 1 from a machine-readable data storage medium, causing the computer to compare the first and second sets of chemical shifts, and causing the computer to identify at least a portion of the atoms that exhibit changes in chemical shifts, wherein the ligand binding site includes the identified atoms. Preferably the papillomavirus E2 protein is encoded by the HPV-18 strain. Preferably the method further includes causing the computer to visually display a spatial arrangement of atoms of the ligand binding site.

Methods disclosed in the present invention for identifying sites offer advantages over other methods known in the art. For example, the present invention preferably provides methods for efficiently identifying binding sites for a wide range of chemically and physically diverse potential ligands.

The term “binding” as used herein, refers to a condition of proximity between a chemical entity or compound, or portions thereof, and the target protein or portions thereof. The association may be non-covalent, wherein the juxtaposition is energetically favored by hydrogen bonding, van der Waals forces, or electrostatic interactions, or it may be covalent. The association may be a static interaction, or an equilibrium may be reached between associated and non-associated species. Preferably, a ligand that binds to a ligand binding site in a DNA-binding and dimerization domain of a papillomavirus E2 protein would also be expected to bind to or interfere with another ligand binding site whose structure defines a shape that falls within an acceptable error.

The term “ligand” as used herein means any chemical entity, compound, or portion thereof, that is capable of binding to a protein.

The term “change in chemical shifts” as used herein means the observation of an increase or decrease in chemical shift for a resonance, an increase or decrease in intensity for a resonance, or the failure to observe a resonance when comparing a resonance of an atom from the spectrum of a mixture of ligand and protein to the resonance of the same atom from the spectrum of the protein without the ligand

### BRIEF DESCRIPTION OF THE DRAWINGS

FIG. 1 is an illustration of the deviations from random coil chemical shifts of ¹³C_{α}resonances (in parts per million (ppm)) with assignments for the DNA-binding and dimerization domain of papillomavirus (strain HPV-18) E2 protein as a function of residue number. Random coil chemical shift values are from Wishart et al., Biochem. Cell Biol., 76:153-63 (1998). Locations of secondary structure according to the X-ray structure of BPV-1, HPV-16 and HPV-31 are shown with α (α-helix) and β (β-sheet).

FIG. 2 is an illustration of the 2-dimensional ¹H-¹⁵N heteronuclear single quantum correlation spectrum with assignments for the DNA-binding and dimerization domain of a 0.84 mM papillomavirus (strain HPV-18) E2 protein at 300° K.

### DETAILED DESCRIPTION

Papillomaviruses are a diverse group of small DNA viruses that infect epithelial cells and cause tumor formation. All of the papillomaviruses encode a DNA-binding protein, E2, that is involved in transcriptional regulation and viral replication. E2 protein consists of a C-terminal DNA-binding and dimerization domain (E2-DBD) and N-terminal transactivation domain, separated by a flexible region. E2-DBD from bovine papillomavirus-1 (BPV-1) has been extensively studied, and the X-ray crystallographic structure of E2-DBD bound to DNA consists of a homodimer that includes an eight-stranded β-barrel and two pairs of α-helices (Hedge et al., Nature, 359:505-12 (1992)). The solution and/or crystal structures of homologous E2-DBDs from human papillomavirus-31 (HPV-31) (Liang et al., Biochemistry, 35:2095-2103 (1996), Bussiere et al., Acta Cryst., D54:1367-76 (1998)) and HPV-16 (Hedge et al., J. Mol. Biol., 284:1479-89 (1998)) have been reported and are similar to BPV-1.

The present invention preferably relates to the E2-DBD from the high risk strain HPV-18. The E2 protein ofHPV-18 represses the expression of the major viral transforming genes E6 and E7 and is a cofactor for the replication protein E1 binding to the origin (Kasukawa et al., J. Virol., 72:8166-73 (1998)). The pivotal role of E2 in transcriptional regulation and viral replication makes it a potential target for antiviral therapy.

E2-DBD of HPV-18 has 55% and 60% sequence identity to HPV-16 and HPV-31, respectively, and binds to the ACCN₆GGT recognition sequence. Preferably, two amino acid sequences are compared using the Blastp program, version 2.0.9, of the BLAST 2 search algorithm, as described by Tatusova et al., FEMS Microbiol Lett 174, 247-50 (1999), and available at http://www.ncbi.nlm.nih.gov/gorf/bl2.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.”

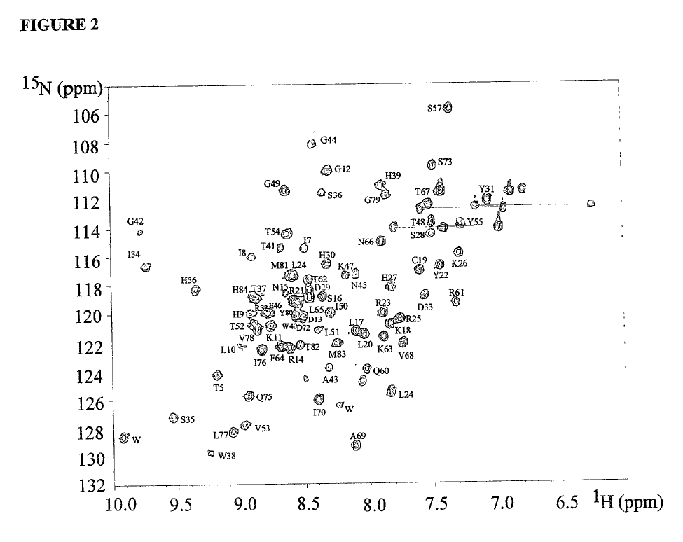

The present invention provides a papillomavirus HPV-18 strain E2 protein DNA-binding domain having the ¹H-¹⁵N heteronuclear single quantum correlation spectrum shown in FIG. 2. Each correlation is labeled as to the residue in the protein from which it arises if that has been determined. The process used to make the assignments is described in the examples. The chemical shifts of all assigned ¹H, ¹³C, and ¹⁵N resonances are listed in Table 1. The resonance assignments presented here provide the basis for determining sites, preferably binding site locations of ligands previously identified by other means. Chemical shift changes induced by addition of ligand to the protein sample are manifested by changes in the appearance of ¹H-¹⁵N HSQC spectra. Correlations that experience the largest ligand-induced chemical shift changes are preferably located near the ligand's binding site. To determine chemical shift changes, the protein ¹H, ¹³C, and ¹⁵N resonances are preferably assigned as extensively as possible.

Preferably, ligand binding sites include identified atoms that exhibit changes in chemical shifts. Preferably the identified atoms include at least one proton that, upon addition of ligand to the protein, either exhibits a change in ¹H chemical shift of at least about 0.04 ppm or is no longer observed. Preferably the identified atoms includes at least one carbon atom that, upon addition of ligand to the protein, either exhibits a change in ¹³C chemical shift of at least about 0.2 ppm or is no longer observed. Preferably the identified atoms include at least one nitrogen atom that, upon addition of ligand to the protein, either exhibits a change in ¹⁵N chemical shift of at least about 0.2 ppm or is no longer observed.

In order that this invention be more fully understood, the following examples are set forth. These examples are for the purpose of illustration only and are not to be construed as limiting the scope of the invention in any way.

### EXAMPLES

The HPV-18 E2 protein consists of 410 amino acids with the DBD residing at the C-terminus (amino acids #329-410). E2-DBD cloning procedures resulted in the addition of methionine before amino acid 329 and six histidine residues after amino acid 410. Amino acid sequencing indicated that the N-terminal des-Met form of the E2-DBD protein was the major species produced.

E2-DBD was over-expressed in BL21 (DE3) E. coli cells using the pSRtac vector. Isotopically labeled samples were prepared in M9 glucose media containing ¹⁵NH₄Cl and unlabeled or U-¹³C-glucose. Cell pellets were lysed with intermittent mechanical disruption with a Tissuemizer (Tekmar Co., Cincinatti, OH). Clarified cell lysates were passed over Ni²⁺-NTA agarose (Qiagen, Inc., Valencia, Calif.), and further purified using Source 30Q anion exchange chromatography (Amersham Pharmacia Biotech, Inc.; Piscataway, N.J.). The resulting E2-DBD exists as a homodimer of molecular weight 20.6 kDa under the conditions used for the NMR experiments.

The NMR samples typically consisted of 0.8 mM protein in buffer containing 20 mM phosphate, 50 mM NaCl, and 1 mM [²H₁₀] dithiothreitol (DTT) at pH 6.5 in 90% ¹H₂O/10% ²H₂O by volume. All NMR spectra were recorded at 27° C. on a Bruker DRX-600 spectrometer (BRUKER NMR, Rheinstetten, Germany) using a 5 mm triple-resonance probe with 3-axis gradients. HNC_{α}, HN(CO)C_{α}, C_{β}C_{α}(CO)NH, H_{β}H_{α}(CO)NH, HNCO and HCCH-total correlation spectroscopy (HCCH-TOCSY) (mixing times 16 and 23 milliseconds) data sets were acquired using gradient-enhanced versions of the pulse sequences. Two-dimensional ¹H-¹⁵N Heteronuclear Single Quantum Correlation (HSQC) and ¹⁵N edited Nuclear Overhauser Effect Spectroscopy-HSQC (NOESY-HSQC) (mixing time 80 milliseconds) spectra were also acquired. Proton chemical shifts were referenced to the ¹H₂O signal at 4.70 parts per million (ppm) (tetramethylsilane (TMS)=0 ppm). The ¹⁵N and ¹³C chemical shifts were referenced indirectly in a manner similar to that known in the art (e.g., Bax et al., J. Magn. Reson., 67:565-69 (1986)). Carrier frequencies were 4.70 ppm for ¹H, 118 ppm for ¹⁵N, 54 ppm for ¹³C_{α}, 40 ppm for aliphatic ¹³C, and 174 ppm for ¹³C′. A combination of water flip-back (e.g., Grzesiek et al., J. Am. Chem. Soc., 115:12593-94 (1993)) and WATERGATE (e.g., Piotto et al., J. Biomol. NMR, 2:661-65 (1992)) techniques were used to eliminate the water resonance. NMR data were processed using NMRPipe and NMRDraw software from Molecular Simulations, Inc. (San Diego, Calif.).

Sequence-specific backbone resonance assignments were accomplished using primarily 3-dimensional HNC_{α}, HN(CO)C_{α}, and C_{β}C_{α}(CO)NH data sets. The ¹³C′ and ¹H_{α}, ¹H_{β} chemical shifts were determined using HNCO and H_{β}H_{α}(CO)NH data sets, respectively. The side chain ¹H and ¹³C spin systems were assigned using the 3-dimensional HCCH-TOCSY experiments.

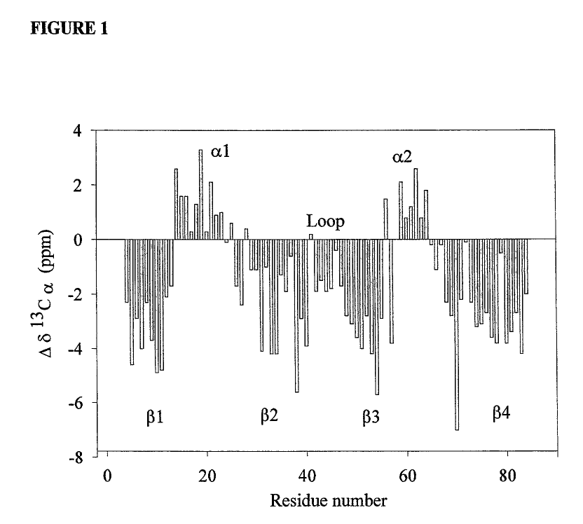

The assigned ¹H-¹⁵N HSQC spectrum of HPV-18 E2-DBD is shown in FIG. 2. Chemical shift values for all ¹H_{N}, ¹H_{α}, ¹³C_{α}, ¹³C_{β}, ¹³C and ¹⁵N_{α} resonances except for the first four residues, the C-terminal five histidine residues, and Glu58 and Thr59 were assigned. Approximately 60% of the side chain ¹H and ¹³C resonances were also assigned. Assigned ¹H, ¹³C, and ¹⁵N chemical shifts are listed in Table 1. The locations of secondary structure in the linear amino acid sequence predicted based on ¹³C_{α} chemical shifts (see Wishart et al., J. Biomol. NMR, 4:171-80 (1994)) are shown in FIG. 1 and are consistent with the crystal structures of BPV-1, HPV-16 and HPV-31.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

TABLE 1
¹H, ¹³C, and ¹⁵N chemical shifts of human papillomavirus E2-DBD.
HA, HB, HG, HD, HE, CA, CB, CG, CD, CE refer to
H_{α}, H_{β}, H_{γ}, H_{δ}, H_{ε},
C_{α}, C_{β}, C_{γ}, C_{δ}, and C_{ε} respectively.

| | | | | |
|---|---|---|---|---|
| #Atom | #RES | RES | ATOMS | ppm |

| | | | | | |
|---|---|---|---|---|---|
| 1 | 4 | THR | HA | H | 5.01 |
| 2 | 4 | THR | HB | H | 3.91 |
| 3 | 4 | THR | HG1 | H | 0.98 |
| 4 | 4 | THR | HG2 | H | 0.98 |
| 5 | 4 | THR | CA | C | 59.95 |
| 6 | 4 | THR | CB | C | 67.75 |
| 7 | 4 | THR | CG2 | C | 19.93 |
| 8 | 5 | THR | H | H | 9.18 |
| 9 | 5 | THR | C | C | 171.68 |
| 10 | 5 | THR | CA | C | 57.48 |
| 11 | 5 | THR | N | N | 124.16 |
| 12 | 6 | PRO | HA | H | 4.73 |
| 13 | 6 | PRO | CA | C | 60.10 |
| 14 | 6 | PRO | CB | C | 29.24 |
| 15 | 7 | ILE | H | H | 8.49 |
| 16 | 7 | ILE | HA | H | 5.85 |
| 17 | 7 | ILE | HB | H | 1.82 |
| 18 | 7 | ILE | HG2 | H | 0.92 |
| 19 | 7 | ILE | HD1 | H | 0.49 |
| 20 | 7 | ILE | C | C | 173.65 |
| 21 | 7 | ILE | CA | C | 57.29 |
| 22 | 7 | ILE | CB | C | 42.10 |
| 23 | 7 | ILE | CG2 | C | 16.79 |
| 24 | 7 | ILE | CD1 | C | 12.90 |
| 25 | 7 | ILE | N | N | 115.39 |
| 26 | 8 | ILE | H | H | 8.90 |
| 27 | 8 | ILE | HA | H | 5.01 |
| 28 | 8 | ILE | HB | H | 1.88 |
| 29 | 8 | ILE | HG2 | H | 0.82 |
| 30 | 8 | ILE | C | C | 174.83 |
| 31 | 8 | ILE | CA | C | 58.93 |
| 32 | 8 | ILE | CB | C | 39.92 |
| 33 | 8 | ILE | CG2 | C | 15.73 |
| 34 | 8 | ILE | N | N | 115.93 |
| 35 | 9 | HIS | H | H | 8.91 |
| 36 | 9 | HIS | HA | H | 5.68 |
| 37 | 9 | HIS | HB2 | H | 2.81 |
| 38 | 9 | HIS | HB3 | H | 2.57 |
| 39 | 9 | HIS | C | C | 173.19 |
| 40 | 9 | HIS | CA | C | 51.27 |
| 41 | 9 | HIS | CB | C | 32.38 |
| 42 | 9 | HIS | N | N | 119.91 |
| 43 | 10 | LEU | H | H | 8.98 |
| 44 | 10 | LEU | HA | H | 5.17 |
| 45 | 10 | LEU | HB2 | H | 1.66 |
| 46 | 10 | LEU | HB3 | H | 0.92 |
| 47 | 10 | LEU | HG | H | 1.47 |
| 48 | 10 | LEU | HD1 | H | 0.82 |
| 49 | 10 | LEU | HD2 | H | 0.71 |
| 50 | 10 | LEU | C | C | 172.40 |
| 51 | 10 | LEU | CA | C | 50.25 |
| 52 | 10 | LEU | CB | C | 40.76 |
| 53 | 10 | LEU | CG | C | 23.68 |
| 54 | 10 | LEU | N | N | 122.16 |
| 55 | 11 | LYS | H | H | 8.76 |
| 56 | 11 | LYS | HA | H | 5.29 |
| 57 | 11 | LYS | HB2 | H | 1.65 |
| 58 | 11 | LYS | HB3 | H | 1.44 |
| 59 | 11 | LYS | HG2 | H | 1.40 |
| 60 | 11 | LYS | HG3 | H | 1.21 |
| 61 | 11 | LYS | HD2 | H | 1.62 |
| 62 | 11 | LYS | HD3 | H | 1.62 |
| 63 | 11 | LYS | HE2 | H | 2.70 |
| 64 | 11 | LYS | HE3 | H | 2.70 |
| 65 | 11 | LYS | C | C | 172.59 |
| 66 | 11 | LYS | CA | C | 51.76 |
| 67 | 11 | LYS | CB | C | 33.58 |
| 68 | 11 | LYS | CG | C | 22.68 |
| 69 | 11 | LYS | CD | C | 27.38 |
| 70 | 11 | LYS | CE | C | 39.54 |
| 71 | 11 | LYS | N | N | 120.73 |
| 72 | 12 | GLY | H | H | 8.30 |
| 73 | 12 | GLY | HA2 | H | 4.43 |
| 74 | 12 | GLY | HA3 | H | 4.19 |
| 75 | 12 | GLY | C | C | 173.46 |
| 76 | 12 | GLY | CA | C | 42.96 |
| 77 | 12 | GLY | N | N | 109.97 |
| 78 | 13 | ASP | H | H | 8.50 |
| 79 | 13 | ASP | HA | H | 4.59 |
| 80 | 13 | ASP | HB2 | H | 2.77 |
| 81 | 13 | ASP | HB3 | H | 2.61 |
| 82 | 13 | ASP | C | C | 168.61 |
| 83 | 13 | ASP | CA | C | 52.23 |
| 84 | 13 | ASP | CB | C | 40.03 |
| 85 | 13 | ASP | N | N | 120.16 |
| 86 | 14 | ARG | H | H | 8.61 |
| 87 | 14 | ARG | HA | H | 3.58 |
| 88 | 14 | ARG | HB2 | H | 1.72 |
| 89 | 14 | ARG | HB3 | H | 1.68 |
| 90 | 14 | ARG | HG2 | H | 1.47 |
| 91 | 14 | ARG | HG3 | H | 1.47 |
| 92 | 14 | ARG | HD2 | H | 3.07 |
| 93 | 14 | ARG | HD3 | H | 3.02 |
| 94 | 14 | ARG | C | C | 174.68 |
| 95 | 14 | ARG | CA | C | 58.64 |
| 96 | 14 | ARG | CB | C | 27.87 |
| 97 | 14 | ARG | CG | C | 26.01 |
| 98 | 14 | ARG | CD | C | 40.85 |
| 99 | 14 | ARG | N | N | 122.34 |
| 100 | 15 | ASN | H | H | 8.64 |
| 101 | 15 | ASN | HA | H | 4.46 |
| 102 | 15 | ASN | HB2 | H | 2.87 |
| 103 | 15 | ASN | HB3 | H | 2.76 |
| 104 | 15 | ASN | C | C | 176.39 |
| 105 | 15 | ASN | CA | C | 54.42 |
| 106 | 15 | ASN | CB | C | 35.59 |
| 107 | 15 | ASN | N | N | 118.46 |
| 108 | 16 | SER | H | H | 8.35 |
| 109 | 16 | SER | HA | H | 3.86 |
| 110 | 16 | SER | HB2 | H | 4.17 |
| 111 | 16 | SER | HB3 | H | 3.63 |
| 112 | 16 | SER | C | C | 175.96 |
| 113 | 16 | SER | CA | C | 59.80 |
| 114 | 16 | SER | CB | C | 59.96 |
| 115 | 16 | SER | N | N | 118.74 |
| 116 | 17 | LEU | H | H | 8.10 |
| 117 | 17 | LEU | HA | H | 3.84 |
| 118 | 17 | LEU | HB2 | H | 1.64 |
| 119 | 17 | LEU | HB3 | H | 1.17 |
| 120 | 17 | LEU | HD1 | H | 0.45 |
| 121 | 17 | LEU | HD2 | H | 0.38 |
| 122 | 17 | LEU | C | C | 175.25 |
| 123 | 17 | LEU | CA | C | 55.37 |
| 124 | 17 | LEU | CB | C | 38.75 |
| 125 | 17 | LEU | CD1 | C | 23.04 |
| 126 | 17 | LEU | CD2 | C | 19.79 |
| 127 | 17 | LEU | N | N | 121.15 |
| 128 | 18 | LYS | H | H | 7.83 |
| 129 | 18 | LYS | HA | H | 3.91 |
| 130 | 18 | LYS | HB2 | H | 1.97 |
| 131 | 18 | LYS | HB3 | H | 1.97 |
| 132 | 18 | LYS | HG2 | H | 1.39 |
| 133 | 18 | LYS | HG3 | H | 1.27 |
| 134 | 18 | LYS | HD2 | H | 1.70 |
| 135 | 18 | LYS | HD3 | H | 1.60 |
| 136 | 18 | LYS | HE2 | H | 2.95 |
| 137 | 18 | LYS | HE3 | H | 2.95 |
| 138 | 18 | LYS | C | C | 175.74 |
| 139 | 18 | LYS | CA | C | 57.85 |
| 140 | 18 | LYS | CB | C | 29.95 |
| 141 | 18 | LYS | CD | C | 27.55 |
| 142 | 18 | LYS | CE | C | 39.77 |
| 143 | 18 | LYS | N | N | 120.70 |
| 144 | 19 | CYS | H | H | 7.59 |
| 145 | 19 | CYS | HA | H | 4.20 |
| 146 | 19 | CYS | HB2 | H | 3.02 |
| 147 | 19 | CYS | HB3 | H | 2.95 |
| 148 | 19 | CYS | C | C | 177.01 |
| 149 | 19 | CYS | CA | C | 60.14 |
| 150 | 19 | CYS | CB | C | 24.32 |
| 151 | 19 | CYS | N | N | 116.91 |
| 152 | 20 | LEU | H | H | 8.03 |
| 153 | 20 | LEU | HA | H | 4.09 |
| 154 | 20 | LEU | HB2 | H | 1.80 |
| 155 | 20 | LEU | HB3 | H | 1.54 |
| 156 | 20 | LEU | HD1 | H | 0.90 |
| 157 | 20 | LEU | HD2 | H | 0.82 |
| 158 | 20 | LEU | C | C | 175.16 |
| 159 | 20 | LEU | CA | C | 55.39 |
| 160 | 20 | LEU | CB | C | 39.82 |
| 161 | 20 | LEU | CD1 | C | 21.58 |
| 162 | 20 | LEU | CD2 | C | 25.17 |
| 163 | 20 | LEU | N | N | 121.40 |
| 164 | 21 | ARG | H | H | 8.58 |
| 165 | 21 | ARG | HA | H | 3.61 |
| 166 | 21 | ARG | HB2 | H | 1.95 |
| 167 | 21 | ARG | C | C | 175.45 |
| 168 | 21 | ARG | CA | C | 58.16 |
| 169 | 21 | ARG | CB | C | 27.32 |
| 170 | 21 | ARG | N | N | 118.96 |
| 171 | 22 | TYR | H | H | 7.43 |
| 172 | 22 | TYR | HA | H | 3.91 |
| 173 | 22 | TYR | C | C | 175.54 |
| 174 | 22 | TYR | CA | C | 59.04 |
| 175 | 22 | TYR | CB | C | 35.58 |
| 176 | 22 | TYR | N | N | 116.61 |
| 177 | 23 | ARG | H | H | 7.88 |
| 178 | 23 | ARG | HA | H | 4.04 |
| 179 | 23 | ARG | HB2 | H | 2.04 |
| 180 | 23 | ARG | HB3 | H | 2.04 |
| 181 | 23 | ARG | HG2 | H | 1.70 |
| 182 | 23 | ARG | HG3 | H | 1.70 |
| 183 | 23 | ARG | HD2 | H | 3.26 |
| 184 | 23 | ARG | HD3 | H | 3.26 |
| 185 | 23 | ARG | C | C | 176.67 |
| 186 | 23 | ARG | CA | C | 57.11 |
| 187 | 23 | ARG | CB | C | 28.01 |
| 188 | 23 | ARG | CG | C | 25.77 |
| 189 | 23 | ARG | CD | C | 41.55 |
| 190 | 23 | ARG | N | N | 119.89 |
| 191 | 24 | LEU | H | H | 8.59 |
| 192 | 24 | LEU | HA | H | 4.18 |
| 193 | 24 | LEU | HB2 | H | 1.89 |
| 194 | 24 | LEU | HB3 | H | 1.46 |
| 195 | 24 | LEU | HD1 | H | 0.80 |
| 196 | 24 | LEU | HD2 | H | 0.60 |
| 197 | 24 | LEU | C | C | 177.05 |
| 198 | 24 | LEU | CA | C | 55.00 |
| 199 | 24 | LEU | CB | C | 38.81 |
| 200 | 24 | LEU | CD1 | C | 21.32 |
| 201 | 24 | LEU | CD2 | C | 22.99 |
| 202 | 24 | LEU | N | N | 117.28 |
| 203 | 25 | ARG | H | H | 7.75 |
| 204 | 25 | ARG | HA | H | 4.26 |
| 205 | 25 | ARG | HB2 | H | 1.91 |
| 206 | 25 | ARG | HB3 | H | 1.91 |
| 207 | 25 | ARG | HG2 | H | 1.82 |
| 208 | 25 | ARG | HG3 | H | 1.82 |
| 209 | 25 | ARG | HD2 | H | 3.11 |
| 210 | 25 | ARG | HD3 | H | 3.11 |
| 211 | 25 | ARG | C | C | 177.46 |
| 212 | 25 | ARG | CA | C | 56.71 |
| 213 | 25 | ARG | CB | C | 27.46 |
| 214 | 25 | ARG | CG | C | 25.14 |
| 215 | 25 | ARG | CD | C | 41.30 |
| 216 | 25 | ARG | N | N | 120.30 |
| 217 | 26 | LYS | H | H | 7.28 |
| 218 | 26 | LYS | HA | H | 4.17 |
| 219 | 26 | LYS | HB2 | H | 1.60 |
| 220 | 26 | LYS | HB3 | H | 1.60 |
| 221 | 26 | LYS | HG2 | H | 1.22 |
| 222 | 26 | LYS | HG3 | H | 1.22 |
| 223 | 26 | LYS | HD2 | H | 1.57 |
| 224 | 26 | LYS | HD3 | H | 1.57 |
| 225 | 26 | LYS | HE2 | H | 2.86 |
| 226 | 26 | LYS | HE3 | H | 2.88 |
| 227 | 26 | LYS | C | C | 175.55 |
| 228 | 26 | LYS | CA | C | 54.84 |
| 229 | 26 | LYS | CB | C | 29.70 |
| 230 | 26 | LYS | CG | C | 22.19 |
| 231 | 26 | LYS | CD | C | 26.73 |
| 232 | 26 | LYS | CE | C | 39.22 |
| 233 | 26 | LYS | N | N | 115.77 |
| 234 | 27 | HIS | H | H | 7.82 |
| 235 | 27 | HIS | HA | H | 5.01 |
| 236 | 27 | HIS | HB2 | H | 3.40 |
| 237 | 27 | HIS | HB3 | H | 2.87 |
| 238 | 27 | HIS | C | C | 174.21 |
| 239 | 27 | HIS | CA | C | 52.56 |
| 240 | 27 | HIS | CB | C | 27.78 |
| 241 | 27 | HIS | N | N | 118.14 |
| 242 | 28 | SER | H | H | 7.50 |
| 243 | 28 | SER | HA | H | 3.46 |
| 244 | 28 | SER | HB2 | H | 3.80 |
| 245 | 28 | SER | HB3 | H | 3.80 |
| 246 | 28 | SER | C | C | 173.31 |
| 247 | 28 | SER | CA | C | 58.63 |
| 248 | 28 | SER | CB | C | 60.65 |
| 249 | 28 | SER | N | N | 114.42 |
| 250 | 29 | ASP | H | H | 8.46 |
| 251 | 29 | ASP | HA | H | 4.42 |
| 252 | 29 | ASP | HB2 | H | 2.43 |
| 253 | 29 | ASP | HB3 | H | 2.21 |
| 254 | 29 | ASP | C | C | 171.83 |
| 255 | 29 | ASP | CA | C | 52.93 |
| 256 | 29 | ASP | CB | C | 37.38 |
| 257 | 29 | ASP | N | N | 118.29 |
| 258 | 30 | HIS | H | H | 8.31 |
| 259 | 30 | HIS | HA | H | 4.90 |
| 260 | 30 | HIS | HB2 | H | 3.75 |
| 261 | 30 | HIS | HB3 | H | 3.33 |
| 262 | 30 | HIS | C | C | 175.04 |
| 263 | 30 | HIS | CA | C | 53.95 |
| 264 | 30 | HIS | CB | C | 29.17 |
| 265 | 30 | HIS | N | N | 116.46 |
| 266 | 31 | TYR | H | H | 7.05 |
| 267 | 31 | TYR | HA | H | 4.57 |
| 268 | 31 | TYR | HB2 | H | 2.58 |
| 269 | 31 | TYR | HB3 | H | 2.58 |
| 270 | 31 | TYR | C | C | 170.71 |
| 271 | 31 | TYR | CA | C | 54.00 |
| 272 | 31 | TYR | CB | C | 37.51 |
| 273 | 31 | TYR | N | N | 112.10 |
| 274 | 32 | ARG | H | H | 8.78 |
| 275 | 32 | ARG | HA | H | 4.24 |
| 276 | 32 | ARG | HB2 | H | 1.90 |
| 277 | 32 | ARG | HB3 | H | 1.90 |
| 278 | 32 | ARG | HG2 | H | 0.50 |
| 279 | 32 | ARG | HG3 | H | 0.50 |
| 280 | 32 | ARG | HD2 | H | 2.44 |
| 281 | 32 | ARG | HD3 | H | 2.25 |
| 282 | 32 | ARG | C | C | 170.17 |
| 283 | 32 | ARG | CA | C | 55.16 |
| 284 | 32 | ARG | CB | C | 27.64 |
| 285 | 32 | ARG | CG | C | 28.32 |
| 286 | 32 | ARG | CD | C | 41.50 |
| 287 | 32 | ARG | N | N | 119.90 |
| 288 | 33 | ASP | H | H | 7.55 |
| 289 | 33 | ASP | HA | H | 4.91 |
| 290 | 33 | ASP | HB2 | H | 2.12 |
| 291 | 33 | ASP | HB3 | H | 1.75 |
| 292 | 33 | ASP | C | C | 171.83 |
| 293 | 33 | ASP | CA | C | 49.82 |
| 294 | 33 | ASP | CB | C | 42.75 |
| 295 | 33 | ASP | N | N | 118.71 |
| 296 | 34 | ILE | H | H | 9.72 |
| 297 | 34 | ILE | HA | H | 5.41 |
| 298 | 34 | ILE | HB | H | 1.31 |
| 299 | 34 | ILE | HG2 | H | 0.91 |
| 300 | 34 | ILE | HD1 | H | 0.45 |
| 301 | 34 | ILE | C | C | 170.37 |
| 302 | 34 | ILE | CA | C | 57.10 |
| 303 | 34 | ILE | CB | C | 39.64 |
| 304 | 34 | ILE | CG2 | C | 17.26 |
| 305 | 34 | ILE | N | N | 116.54 |
| 306 | 35 | SER | H | H | 9.53 |
| 307 | 35 | SER | HA | H | 5.10 |
| 308 | 35 | SER | HB2 | H | 3.98 |
| 309 | 35 | SER | HB3 | H | 3.98 |
| 310 | 35 | SER | C | C | 173.41 |
| 311 | 35 | SER | CA | C | 56.93 |
| 312 | 35 | SER | CB | C | 64.81 |
| 313 | 35 | SER | N | N | 127.07 |
| 314 | 36 | SER | H | H | 8.34 |
| 315 | 36 | SER | HA | H | 4.17 |
| 316 | 36 | SER | HB2 | H | 2.94 |
| 317 | 36 | SER | HB3 | H | 2.94 |
| 318 | 36 | SER | C | C | 171.93 |
| 319 | 36 | SER | CA | C | 56.27 |
| 320 | 36 | SER | CB | C | 61.52 |
| 321 | 36 | SER | N | N | 111.52 |
| 322 | 37 | THR | H | H | 8.87 |
| 323 | 37 | THR | HA | H | 4.42 |
| 324 | 37 | THR | HB | H | 3.98 |
| 325 | 37 | THR | HG2 | H | 0.99 |
| 326 | 37 | THR | C | C | 172.22 |
| 327 | 37 | THR | CA | C | 61.50 |
| 328 | 37 | THR | CB | C | 66.25 |
| 329 | 37 | THR | CG2 | C | 20.38 |
| 330 | 37 | THR | N | N | 118.94 |
| 331 | 38 | TRP | H | H | 9.25 |
| 332 | 38 | TRP | HA | H | 4.75 |
| 333 | 38 | TRP | HB2 | H | 2.54 |
| 334 | 38 | TRP | HB3 | H | 2.54 |
| 335 | 38 | TRP | C | C | 172.46 |
| 336 | 38 | TRP | CA | C | 52.15 |
| 337 | 38 | TRP | CB | C | 29.53 |
| 338 | 38 | TRP | N | N | 129.61 |
| 339 | 39 | HIS | H | H | 7.89 |
| 340 | 39 | HIS | HA | H | 4.44 |
| 341 | 39 | HIS | HB2 | H | 2.43 |
| 342 | 39 | HIS | HB3 | H | 2.43 |
| 343 | 39 | HIS | C | C | 169.88 |
| 344 | 39 | HIS | CA | C | 52.09 |
| 345 | 39 | HIS | CB | C | 30.38 |
| 346 | 40 | TRP | H | H | 8.56 |
| 347 | 40 | TRP | HA | H | 5.08 |
| 348 | 40 | TRP | HB2 | H | 3.64 |
| 349 | 40 | TRP | HB3 | H | 2.87 |
| 350 | 40 | TRP | C | C | 171.67 |
| 351 | 40 | TRP | CA | C | 53.85 |
| 352 | 40 | TRP | CB | C | 27.77 |
| 353 | 40 | TRP | N | N | 120.03 |
| 354 | 41 | THR | H | H | 8.67 |
| 355 | 41 | THR | HA | H | 4.42 |
| 356 | 41 | THR | HB | H | 3.92 |
| 357 | 41 | THR | HG2 | H | 0.99 |
| 358 | 41 | THR | C | C | 175.17 |
| 359 | 41 | THR | CA | C | 62.27 |
| 360 | 41 | THR | CB | C | 67.99 |
| 361 | 41 | THR | CG2 | C | 20.38 |
| 362 | 41 | THR | N | N | 115.31 |
| 363 | 42 | GLY | H | H | 9.77 |
| 364 | 42 | GLY | HA2 | H | 4.03 |
| 365 | 42 | GLY | HA3 | H | 4.03 |
| 366 | 42 | GLY | C | C | 173.88 |
| 367 | 42 | GLY | CA | C | 43.28 |
| 368 | 42 | GLY | N | N | 114.16 |
| 369 | 43 | ALA | H | H | 8.31 |
| 370 | 43 | ALA | HA | H | 4.32 |
| 371 | 43 | ALA | HB | H | 1.39 |
| 372 | 43 | ALA | C | C | 172.26 |
| 373 | 43 | ALA | CA | C | 50.72 |
| 374 | 43 | ALA | CB | C | 16.84 |
| 375 | 43 | ALA | N | N | 123.70 |
| 376 | 44 | GLY | H | H | 8.42 |
| 377 | 44 | GLY | HA2 | H | 4.10 |
| 378 | 44 | GLY | HA3 | H | 3.91 |
| 379 | 44 | GLY | C | C | 176.29 |
| 380 | 44 | GLY | CA | C | 43.25 |
| 381 | 44 | GLY | N | N | 108.16 |
| 382 | 45 | ASN | HA | H | 4.75 |
| 383 | 45 | ASN | HB2 | H | 2.93 |
| 384 | 45 | ASN | HB3 | H | 2.75 |
| 385 | 45 | ASN | C | C | 172.12 |
| 386 | 45 | ASN | CA | C | 50.98 |
| 387 | 45 | ASN | CB | C | 37.51 |
| 388 | 45 | ASN | N | N | 117.19 |
| 389 | 46 | GLU | H | H | 8.81 |
| 390 | 46 | GLU | HA | H | 3.98 |
| 391 | 46 | GLU | HB2 | H | 1.93 |
| 392 | 46 | GLU | HB3 | H | 1.87 |
| 393 | 46 | GLU | HG2 | H | 2.14 |
| 394 | 46 | GLU | HG3 | H | 2.14 |
| 395 | 46 | GLU | C | C | 173.36 |
| 396 | 46 | GLU | CA | C | 55.97 |
| 397 | 46 | GLU | CB | C | 27.17 |
| 398 | 46 | GLU | CG | C | 33.95 |
| 399 | 46 | GLU | N | N | 119.81 |
| 400 | 47 | LYS | H | H | 8.17 |
| 401 | 47 | LYS | HA | H | 4.19 |
| 402 | 47 | LYS | HB2 | H | 1.94 |
| 403 | 47 | LYS | HB3 | H | 1.76 |
| 404 | 47 | LYS | HG2 | H | 1.40 |
| 405 | 47 | LYS | HG3 | H | 1.33 |
| 406 | 47 | LYS | HD2 | H | 1.60 |
| 407 | 47 | LYS | HD3 | H | 1.60 |
| 408 | 47 | LYS | HE2 | H | 2.94 |
| 409 | 47 | LYS | HE3 | H | 2.94 |
| 410 | 47 | LYS | C | C | 174.43 |
| 411 | 47 | LYS | CA | C | 54.79 |
| 412 | 47 | LYS | CB | C | 30.57 |
| 413 | 47 | LYS | CG | C | 22.93 |
| 414 | 47 | LYS | CD | C | 26.73 |
| 415 | 47 | LYS | CE | C | 39.80 |
| 416 | 47 | LYS | N | N | 117.28 |
| 417 | 48 | THR | H | H | 7.49 |
| 418 | 48 | THR | HA | H | 4.37 |
| 419 | 48 | THR | HB | H | 3.99 |
| 420 | 48 | THR | HG1 | H | 1.05 |
| 421 | 48 | THR | HG2 | H | 1.05 |
| 422 | 48 | THR | C | C | 174.80 |
| 423 | 48 | THR | CA | C | 59.28 |
| 424 | 48 | THR | CB | C | 68.23 |
| 425 | 48 | THR | CG2 | C | 19.72 |
| 426 | 48 | THR | N | N | 113.55 |
| 427 | 49 | GLY | H | H | 8.64 |
| 428 | 49 | GLY | HA2 | H | 4.28 |
| 429 | 49 | GLY | HA3 | H | 3.05 |
| 430 | 49 | GLY | C | C | 171.67 |
| 431 | 49 | GLY | CA | C | 42.01 |
| 432 | 49 | GLY | N | N | 111.32 |
| 433 | 50 | ILE | H | H | 8.29 |
| 434 | 50 | ILE | HA | H | 4.53 |
| 435 | 50 | ILE | HB | H | −1.31 |
| 436 | 50 | ILE | HG2 | H | −0.31 |
| 437 | 50 | ILE | C | C | 168.12 |
| 438 | 50 | ILE | CA | C | 57.68 |
| 439 | 50 | ILE | CB | C | 37.82 |
| 440 | 50 | ILE | N | N | 119.88 |
| 441 | 51 | LEU | H | H | 8.39 |
| 442 | 51 | LEU | HA | H | 4.30 |
| 443 | 51 | LEU | HB2 | H | 1.44 |
| 444 | 51 | LEU | HB3 | H | 1.24 |
| 445 | 51 | LEU | HG | H | 1.44 |
| 446 | 51 | LEU | HD1 | H | 0.67 |
| 447 | 51 | LEU | C | C | 171.45 |
| 448 | 51 | LEU | CA | C | 51.06 |
| 449 | 51 | LEU | CB | C | 44.03 |
| 450 | 51 | LEU | CG | C | 24.41 |
| 451 | 51 | LEU | CD1 | C | 23.46 |
| 452 | 51 | LEU | N | N | 120.99 |
| 453 | 52 | THR | H | H | 8.89 |
| 454 | 52 | THR | HA | H | 5.22 |
| 455 | 52 | THR | HB | H | 3.52 |
| 456 | 52 | THR | HG2 | H | 1.30 |
| 457 | 52 | THR | C | C | 173.14 |
| 458 | 52 | THR | CA | C | 59.30 |
| 459 | 52 | THR | CB | C | 72.25 |
| 460 | 52 | THR | CG2 | C | 22.71 |
| 461 | 52 | THR | N | N | 120.58 |
| 462 | 53 | VAL | H | H | 8.97 |
| 463 | 53 | VAL | HA | H | 4.71 |
| 464 | 53 | VAL | HB | H | 1.65 |
| 465 | 53 | VAL | HG1 | H | 0.43 |
| 466 | 53 | VAL | HG2 | H | 0.16 |
| 467 | 53 | VAL | C | C | 170.60 |
| 468 | 53 | VAL | CA | C | 58.06 |
| 469 | 53 | VAL | CB | C | 31.00 |
| 470 | 53 | VAL | CG1 | C | 18.20 |
| 471 | 53 | VAL | CG2 | C | 20.37 |
| 472 | 53 | VAL | N | N | 127.66 |
| 473 | 54 | THR | H | H | 8.63 |
| 474 | 54 | THR | HA | H | 5.00 |
| 475 | 54 | THR | HB | H | 3.87 |
| 476 | 54 | THR | HG2 | H | 1.03 |
| 477 | 54 | THR | C | C | 172.93 |
| 478 | 54 | THR | CA | C | 56.41 |
| 479 | 54 | THR | CB | C | 68.61 |
| 480 | 54 | THR | CG2 | C | 19.60 |
| 481 | 54 | THR | N | N | 114.36 |
| 482 | 55 | TYR | H | H | 7.26 |
| 483 | 55 | TYR | HA | H | 4.61 |
| 484 | 55 | TYR | HB2 | H | 3.55 |
| 485 | 55 | TYR | HB3 | H | 3.55 |
| 486 | 55 | TYR | C | C | 171.06 |
| 487 | 55 | TYR | CA | C | 55.21 |
| 488 | 55 | TYR | CB | C | 40.88 |
| 489 | 55 | TYR | N | N | 113.74 |
| 490 | 56 | HIS | H | H | 9.34 |
| 491 | 56 | HIS | HA | H | 4.42 |
| 492 | 56 | HIS | HB2 | H | 3.08 |
| 493 | 56 | HIS | HB3 | H | 2.81 |
| 494 | 56 | HIS | C | C | 173.18 |
| 495 | 56 | HIS | CA | C | 56.49 |
| 496 | 56 | HIS | CB | C | 29.81 |
| 497 | 56 | HIS | N | N | 118.21 |
| 498 | 57 | SER | H | H | 7.34 |
| 499 | 57 | SER | C | C | 173.49 |
| 500 | 57 | SER | CA | C | 54.41 |
| 501 | 57 | SER | N | N | 105.78 |
| 502 | 59 | THR | HA | H | 3.91 |
| 503 | 59 | THR | HB | H | 4.07 |
| 504 | 59 | THR | HG2 | H | 1.20 |
| 505 | 59 | THR | CA | C | 64.19 |
| 506 | 59 | THR | CB | C | 66.34 |
| 507 | 59 | THR | CG2 | C | 18.99 |
| 508 | 60 | GLN | H | H | 8.02 |
| 509 | 60 | GLN | HA | H | 4.06 |
| 510 | 60 | GLN | HB2 | H | 2.09 |
| 511 | 60 | GLN | HB3 | H | 2.09 |
| 512 | 60 | GLN | HG2 | H | 3.26 |
| 513 | 60 | GLN | HG3 | H | 3.26 |
| 514 | 60 | GLN | C | C | 174.20 |
| 515 | 60 | GLN | CA | C | 56.90 |
| 516 | 60 | GLN | CB | C | 27.27 |
| 517 | 60 | GLN | CG | C | 41.55 |
| 518 | 60 | GLN | N | N | 123.81 |
| 519 | 61 | ARG | H | H | 7.31 |
| 520 | 61 | ARG | HA | H | 2.99 |
| 521 | 61 | ARG | HB2 | H | 1.70 |
| 522 | 61 | ARG | HB3 | H | 1.70 |
| 523 | 61 | ARG | C | C | 175.22 |
| 524 | 61 | ARG | CA | C | 57.25 |
| 525 | 61 | ARG | CB | C | 27.77 |
| 526 | 61 | ARG | N | N | 119.25 |
| 527 | 62 | THR | H | H | 8.47 |
| 528 | 62 | THR | HA | H | 3.71 |
| 529 | 62 | THR | HB | H | 4.21 |
| 530 | 62 | THR | HG2 | H | 1.16 |
| 531 | 62 | THR | C | C | 174.94 |
| 532 | 62 | THR | CA | C | 64.67 |
| 533 | 62 | THR | CB | C | 66.46 |
| 534 | 62 | THR | CG2 | C | 19.65 |
| 535 | 62 | THR | N | N | 117.57 |
| 536 | 63 | LYS | H | H | 7.88 |
| 537 | 63 | LYS | HA | H | 4.05 |
| 538 | 63 | LYS | HB2 | H | 1.90 |
| 539 | 63 | LYS | HB3 | H | 1.90 |
| 540 | 63 | LYS | HG2 | H | 1.29 |
| 541 | 63 | LYS | HG3 | H | 1.29 |
| 542 | 63 | LYS | HD2 | H | 1.59 |
| 543 | 63 | LYS | HD3 | H | 1.59 |
| 544 | 63 | LYS | HE2 | H | 2.84 |
| 545 | 63 | LYS | HE3 | H | 2.79 |
| 546 | 63 | LYS | C | C | 173.47 |
| 547 | 63 | LYS | CA | C | 57.28 |
| 548 | 63 | LYS | CB | C | 29.34 |
| 549 | 63 | LYS | CG | C | 22.63 |
| 550 | 63 | LYS | CD | C | 26.76 |
| 551 | 63 | LYS | CE | C | 39.80 |
| 552 | 63 | LYS | N | N | 121.56 |
| 553 | 64 | PHE | HA | H | 3.94 |
| 554 | 64 | PHE | HB2 | H | 3.75 |
| 555 | 64 | PHE | HB3 | H | 3.75 |
| 556 | 64 | PHE | C | C | 177.53 |
| 557 | 64 | PHE | CA | C | 59.77 |
| 558 | 64 | PHE | CB | C | 35.86 |
| 559 | 64 | PHE | N | N | 122.19 |
| 560 | 65 | LEU | H | H | 8.46 |
| 561 | 65 | LEU | HA | H | 4.03 |
| 562 | 65 | LEU | HB2 | H | 1.92 |
| 563 | 65 | LEU | HB3 | H | 1.33 |
| 564 | 65 | LEU | HD1 | H | 0.67 |
| 565 | 65 | LEU | HD2 | H | 0.48 |
| 566 | 65 | LEU | C | C | 174.91 |
| 567 | 65 | LEU | CA | C | 54.86 |
| 568 | 65 | LEU | CB | C | 39.32 |
| 569 | 65 | LEU | CD1 | C | 19.30 |
| 570 | 65 | LEU | CD2 | C | 22.91 |
| 571 | 65 | LEU | N | N | 118.84 |
| 572 | 66 | ASN | H | H | 7.89 |
| 573 | 66 | ASN | HA | H | 4.72 |
| 574 | 66 | ASN | HB2 | H | 2.84 |
| 575 | 66 | ASN | HB3 | H | 2.76 |
| 576 | 66 | ASN | C | C | 176.34 |
| 577 | 66 | ASN | CA | C | 51.67 |
| 578 | 66 | ASN | CB | C | 37.26 |
| 579 | 66 | ASN | N | N | 114.93 |
| 580 | 67 | THR | H | H | 7.52 |
| 581 | 67 | THR | HA | H | 4.25 |
| 582 | 67 | THR | HB | H | 3.74 |
| 583 | 67 | THR | HG2 | H | 0.96 |
| 584 | 67 | THR | C | C | 173.66 |
| 585 | 67 | THR | CA | C | 61.85 |
| 586 | 67 | THR | CB | C | 68.91 |
| 587 | 67 | THR | CG2 | C | 18.92 |
| 588 | 67 | THR | N | N | 112.40 |
| 589 | 68 | VAL | H | H | 7.73 |
| 590 | 68 | VAL | HA | H | 3.39 |
| 591 | 68 | VAL | HB | H | 1.05 |
| 592 | 68 | VAL | HG1 | H | 0.16 |
| 593 | 68 | VAL | HG2 | H | −0.12 |
| 594 | 68 | VAL | C | C | 171.61 |
| 595 | 68 | VAL | CA | C | 60.07 |
| 596 | 68 | VAL | CB | C | 29.25 |
| 597 | 68 | VAL | CG1 | C | 18.45 |
| 598 | 68 | VAL | CG2 | C | 17.60 |
| 599 | 68 | VAL | N | N | 122.00 |
| 600 | 69 | ALA | H | H | 8.12 |
| 601 | 69 | ALA | HA | H | 4.23 |
| 602 | 69 | ALA | HB | H | 1.19 |
| 603 | 69 | ALA | C | C | 172.02 |
| 604 | 69 | ALA | CA | C | 49.53 |
| 605 | 69 | ALA | CB | C | 15.99 |
| 606 | 69 | ALA | N | N | 129.17 |
| 607 | 70 | ILE | H | H | 8.40 |
| 608 | 70 | ILE | C | C | 174.04 |
| 609 | 70 | ILE | CA | C | 54.26 |
| 610 | 70 | ILE | N | N | 125.89 |
| 611 | 71 | PRO | HA | H | 4.43 |
| 612 | 71 | PRO | HB3 | H | 1.92 |
| 613 | 71 | PRO | HG2 | H | 3.83 |
| 614 | 71 | PRO | HG3 | H | 3.35 |
| 615 | 71 | PRO | CA | C | 60.85 |
| 616 | 71 | PRO | CB | C | 30.38 |
| 617 | 71 | PRO | CG | C | 25.23 |
| 618 | 72 | ASP | H | H | 8.56 |
| 619 | 72 | ASP | HA | H | 4.19 |
| 620 | 72 | ASP | HB2 | H | 2.65 |
| 621 | 72 | ASP | HB3 | H | 2.65 |
| 622 | 72 | ASP | C | C | 174.61 |
| 623 | 72 | ASP | CA | C | 53.85 |
| 624 | 72 | ASP | CB | C | 38.07 |
| 625 | 72 | ASP | N | N | 120.03 |
| 626 | 73 | SER | H | H | 7.48 |
| 627 | 73 | SER | HA | H | 4.26 |
| 628 | 73 | SER | HB2 | H | 4.07 |
| 629 | 73 | SER | HB3 | H | 3.83 |
| 630 | 73 | SER | C | C | 173.98 |
| 631 | 73 | SER | CA | C | 55.90 |
| 632 | 73 | SER | CB | C | 60.58 |
| 633 | 73 | SER | N | N | 109.69 |
| 634 | 74 | VAL | H | H | 7.83 |
| 635 | 74 | VAL | HA | H | 4.45 |
| 636 | 74 | VAL | HB | H | 1.99 |
| 637 | 74 | VAL | HG1 | H | 0.66 |
| 638 | 74 | VAL | HG2 | H | 0.62 |
| 639 | 74 | VAL | C | C | 171.92 |
| 640 | 74 | VAL | CA | C | 59.08 |
| 641 | 74 | VAL | CB | C | 30.98 |
| 642 | 74 | VAL | CG1 | C | 20.02 |
| 643 | 74 | VAL | CG2 | C | 20.02 |
| 644 | 74 | VAL | N | N | 125.42 |
| 645 | 75 | GLN | H | H | 8.94 |
| 646 | 75 | GLN | HA | H | 4.45 |
| 647 | 75 | GLN | HB2 | H | 2.03 |
| 648 | 75 | GLN | HB3 | H | 1.90 |
| 649 | 75 | GLN | HG2 | H | 2.43 |
| 650 | 75 | GLN | HG3 | H | 2.23 |
| 651 | 75 | GLN | C | C | 172.04 |
| 652 | 75 | GLN | CA | C | 53.00 |
| 653 | 75 | GLN | CB | C | 28.74 |
| 654 | 75 | GLN | CG | C | 32.19 |
| 655 | 75 | GLN | N | N | 125.65 |
| 656 | 76 | ILE | H | H | 8.83 |
| 657 | 76 | ILE | HA | H | 4.63 |
| 658 | 76 | ILE | HB | H | 1.88 |
| 659 | 76 | ILE | HG2 | H | 0.67 |
| 660 | 76 | ILE | C | C | 172.76 |
| 661 | 76 | ILE | CA | C | 58.71 |
| 662 | 76 | ILE | CB | C | 37.76 |
| 663 | 76 | ILE | CG2 | C | 15.81 |
| 664 | 76 | ILE | N | N | 122.43 |
| 665 | 77 | LEU | H | H | 9.07 |
| 666 | 77 | LEU | HA | H | 5.04 |
| 667 | 77 | LEU | HB2 | H | 1.65 |
| 668 | 77 | LEU | HB3 | H | 1.30 |
| 669 | 77 | LEU | HG | H | 1.43 |
| 670 | 77 | LEU | HD1 | H | 0.74 |
| 671 | 77 | LEU | HD2 | H | 0.60 |
| 672 | 77 | LEU | C | C | 172.98 |
| 673 | 77 | LEU | CA | C | 51.54 |
| 674 | 77 | LEU | CB | C | 41.98 |
| 675 | 77 | LEU | CG | C | 25.94 |
| 676 | 77 | LEU | CD1 | C | 22.69 |
| 677 | 77 | LEU | CD2 | C | 22.12 |
| 678 | 77 | LEU | N | N | 128.16 |
| 679 | 78 | VAL | H | H | 8.87 |
| 680 | 78 | VAL | HA | H | 4.38 |
| 681 | 78 | VAL | HB | H | 1.55 |
| 682 | 78 | VAL | HG1 | H | 0.71 |
| 683 | 78 | VAL | HG2 | H | 0.71 |
| 684 | 78 | VAL | C | C | 173.14 |
| 685 | 78 | VAL | CA | C | 58.45 |
| 686 | 78 | VAL | CB | C | 32.33 |
| 687 | 78 | VAL | CG1 | C | 19.09 |
| 688 | 78 | VAL | CG2 | C | 19.09 |
| 689 | 78 | VAL | N | N | 121.05 |
| 690 | 79 | GLY | H | H | 7.86 |
| 691 | 79 | GLY | HA2 | H | 5.08 |
| 692 | 79 | GLY | HA3 | H | 4.08 |
| 693 | 79 | GLY | C | C | 172.86 |
| 694 | 79 | GLY | CA | C | 44.62 |
| 695 | 79 | GLY | N | N | 111.73 |
| 696 | 80 | TYR | H | H | 8.54 |
| 697 | 80 | TYR | HA | H | 5.37 |
| 698 | 80 | TYR | HB2 | H | 2.99 |
| 699 | 80 | TYR | HB3 | H | 2.61 |
| 700 | 80 | TYR | C | C | 169.75 |
| 701 | 80 | TYR | CA | C | 54.23 |
| 702 | 80 | TYR | CB | C | 40.30 |
| 703 | 80 | TYR | N | N | 119.24 |
| 704 | 81 | MET | H | H | 8.60 |
| 705 | 81 | MET | HA | H | 5.35 |
| 706 | 81 | MET | HB2 | H | 1.94 |
| 707 | 81 | MET | HB3 | H | 1.94 |
| 708 | 81 | MET | HG2 | H | 2.55 |
| 709 | 81 | MET | HG3 | H | 2.50 |
| 710 | 81 | MET | C | C | 171.31 |
| 711 | 81 | MET | CA | C | 51.86 |
| 712 | 81 | MET | CB | C | 34.66 |
| 713 | 81 | MET | CG | C | 29.09 |
| 714 | 81 | MET | N | N | 117.15 |
| 715 | 82 | THR | H | H | 8.53 |
| 716 | 82 | THR | HA | H | 4.98 |
| 717 | 82 | THR | HB | H | 3.51 |
| 718 | 82 | THR | HG2 | H | 1.06 |
| 719 | 82 | THR | C | C | 172.03 |
| 720 | 82 | THR | CA | C | 59.38 |
| 721 | 82 | THR | CB | C | 68.52 |
| 722 | 82 | THR | CG2 | C | 19.60 |
| 723 | 82 | THR | N | N | 122.12 |
| 724 | 83 | MET | H | H | 8.25 |
| 725 | 83 | MET | HA | H | 5.19 |
| 726 | 83 | MET | C | C | 170.95 |
| 727 | 83 | MET | CA | C | 51.06 |
| 728 | 83 | MET | CB | C | 33.27 |
| 729 | 83 | MET | N | N | 122.01 |
| 730 | 84 | HIS | H | H | 8.90 |
| 731 | 84 | HIS | C | C | 173.02 |
| 732 | 84 | HIS | CA | C | 53.04 |
| 733 | 84 | HIS | N | N | 118.65 |

What is claimed is:

1. A nuclear magnetic resonance method for identifying a site in a DNA-binding and dimerization domain of a papillomavirus E2 protein, the method comprising:
providing a first set of chemical shifts for atoms of a mixture comprising a ligand and the papillomavirus E2 protein;
comparing the first set of chemical shifts to a second set of chemical shifts as listed in Table 1; and
identifying at least a portion of the atoms that exhibit changes in chemical shifts, wherein the site comprises the identified atoms.

2. The method of claim 1 wherein providing the first set of chemical shifts comprises:
providing a mixture of the ligand and the papillomavirus E2 protein;
allowing the ligand to interact with the papillomavirus E2 protein;
obtaining a nuclear magnetic resonance spectrum of the mixture; and
measuring chemical shifts of atoms from the spectrum.

3. The method of claim 2 wherein allowing the ligand to interact comprises allowing the ligand and the protein to reach a binding equilibrium.

4. The method of claim 1 wherein the site is a ligand binding site.

5. The method of claim 1 wherein the papillomavirus E2 protein is encoded by the HPV-18 strain.

6. The method of claim 1 wherein identifying at least a portion of the atoms comprises indentifying at least one proton that either exhibits a change in ¹H chemical shift of at least about 0.04 ppm or is no longer observed.

7. The method of claim 1 wherein identifying at least a portion of the atoms comprises identifying at least one carbon atom that either exhibits a change in ¹³C chemical shift of at least about 0.2 ppm or is no longer observed.

8. The method of claim 1 wherein identifying at least a portion of the atoms comprises identifying at least one nitrogen atom that either exhibits a change in ¹⁵N chemical shift of at least about 0.2 ppm or is no longer observed.

9. A nuclear magnetic resonance method for identifying a site in a DNA-binding and dimerization domain of a papillomavirus E2 protein, the method comprising:
providing a first ¹H-¹⁵N heteronuclear single quantum correlation spectrum of a mixture comprising a ligand and the papillomavirus E2 protein;
comparing the first ¹H-¹⁵N heteronuclear single quantum correlation spectrum to a second ¹H-¹⁵N heteronuclear single quantum correlation spectrum as illustrated in FIG. 2; and
identifying at least a portion of the amino acids having atoms that exhibit changes in chemical shifts, wherein the site comprises the identified amino acids.

10. The method of claim 9 wherein providing the first spectrum comprises:
providing a mixture of the ligand and the papillomavirus E2 protein;
allowing the ligand to interact with the papillomavirus E2 protein; and
obtaining a ¹H-¹⁵N heteronuclear single quantum correlation spectrum of the mixture.

11. The method of claim 10 wherein allowing the ligand to interact comprises allowing the ligand and the protein to reach a binding equilibrium.

12. The method of claim 9 wherein the site is a ligand binding site.

13. The method of claim 9 wherein the papillomavirus E2 protein is encoded by the HPV-18 strain.

14. The method of claim 9 wherein identifying at least a portion of the amino acids comprises identifying at least one amino acid having a proton that either exhibits a change in ¹H chemical shift of at least about 0.04 ppm or is no longer observed.

15. The method of claim 9 wherein identifying at least a portion of the amino acids comprises identifying at least one amino acid having a nitrogen atom that either exhibits a change in ¹⁵N chemical shift of at least about 0.2 ppm or is no longer observed.

16. A machine-readable data storage medium comprising a data storage material encoded with nuclear magnetic resonance chemical shifts as listed in Table 1, wherein when a first set of chemical shifts is provided, the chemical shifts encoded on the data storage material are capable of being read by the machine to create a second set of chemical shifts, and the machine having programmed instructions that are capable of causing the machine to compare the first and second sets of chemical shifts to arrive at structural information.

17. A computer-assisted method for identifying a ligand binding site in a DNA-binding and dimerization domain of a papillomavirus E2 protein, the method comprising:
providing a first set of nuclear magnetic resonance chemical shifts for atoms of a mixture comprising the ligand and the papillomavirus E2 protein;
causing the first set of chemical shifts to be entered into memory of a computer;
causing the computer to read a second set of chemical shifts as listed in Table 1 from a machine-readable data storage medium;
causing the computer to compare the first and second sets of chemical shifts; and
causing the computer to identify at least a portion of the atoms that exhibit changes in chemical shifts, wherein the ligand binding site comprises the identified atoms.

18. The method of claim 17 wherein the papillomavirus E2 protein is encoded by the HPV-18 strain.

19. The method of claim 17 wherein causing the computer to identify at least a portion of the atoms comprises causing the computer to identify at least one proton that either exhibits a change in ¹H chemical shift of at least about 0.04 ppm or is no longer observed.

20. The method of claim 17 wherein causing the computer to identify at least a portion of the atoms comprises causing the computer to identify at least one carbon atom that either exhibits a change in ¹³C chemical shift of at least about 0.2 ppm or is no longer observed.

21. The method of claim 17 wherein causing the computer to identify at least a portion of the atoms comprises causing the computer to identify a nitrogen atom that either exhibits a change in ¹⁵N chemical shift of at least about 0.2 ppm or is no longer observed.

22. The method of claim 17 further comprising causing the computer to visually display a spatial arrangement of atoms of the ligand binding site.
